(19) United States
(12) Patent Application Publication
(10) Pub. No.: US 2018/0370918 A1
(43) Pub. Date: Dec. 27, 2018

(54) SORAFENIB HEMI-P-TOSYLATE MONOHYDRATE CRYSTAL AND PREPARATION PROCESS THEREOF

(71) Applicant: CHIA TAI TIANQING PHARMACEUTICAL GROUP CO., LTD., Lianyungang, Jiangsu (CN)

(72) Inventors: Shufeng WU, Lianyungang, Jiangsu (CN); Aiming ZHANG, Lianyungang, Jiangsu (CN); Xiquan ZHANG, Lianyungang, Jiangsu (CN); Xueyan ZHU, Shanghai (CN)

(21) Appl. No.: 16/062,030

(22) PCT Filed: Sep. 23, 2016
(86) PCT No.: PCT/CN2016/099871

§ 371 Date: Jun. 13, 2018

(30) Foreign Application Priority Data
Dec. 14, 2015 (CN) 201510927428.5

(51) Int. Cl. C07D 213/81; A61P 35/00

(52) U.S. Cl. CPC C07D 213/81; A61P 35/00

(57) ABSTRACT

The present invention relates to the field of medicinal technology, and in particular, to sorafenib hemi-p-tosylate monohydrate crystal and preparation process thereof. The crystal has diffraction peaks occurring at 2θ angle of about 5.62, 6.67, 8.05, 9.06, 9.63, 9.91, 10.95, 11.25, 13.48, 14.00, 14.60, 15.08, 15.75, 16.20, 16.62, 16.80, 17.23, 18.40, 18.97, 19.32, 19.82, 20.49, 20.74, 21.51, 22.56, 22.86, 23.37, 23.71, 24.20, 24.71, 24.97, 25.54, 25.80, 26.18, 27.14, 27.48, and 28.29 degree in a X-ray powder diffraction pattern, and some advantages, such as a high stability, a low hygroscopicity and the like.

### TECHNICAL FIELD

The present invention relates to the field of medicinal technology, and in particular, to sorafenib hemi-p-tosylate monohydrate crystal and preparation process thereof.

### BACKGROUND TECHNOLOGY

Sorafenib tosylate has the structure represented by formula (1), and its chemical name is 4-{4-[3-(4-chloro-3-(trifluoromethyl)-phenyl)-ureido]-phenoxy}-N-methyl pyridine-2-carboxamide 4-methylbenzenesulfonate. Sorafenib is developed and marketed by Bayer and Onyx. Sorafenib is an oral small-molecule kinase inhibitor for inhibiting a cell growth, is used for treating renal cell carcinoma (RCC) and unresectable hepatocellular carcinoma (HCC).

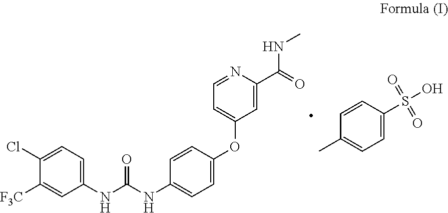 [SMILES: CNC(=O)c1cc(Oc2ccc(NC(=O)Nc3ccc(Cl)c(C(F)(F)F)c3)cc2)ccn1 ; Cc1ccc(S(=O)(=O)O)cc1]

CN101052619, WO2009034308 and US20130005980 disclose a process for preparing sorafenib tosylate. CN101065360 discloses three crystals of sorafenib tosylate (I, II, and III), and their corresponding preparation processes. In order to obtain a stable crystal, a crystal transformation with stirring at a high temperature or for a long period of time is needed, and the resulting crystal forms have a low degree of crystallinity. Therefore, such processes cannot meet the requirement for a large-scale industrial production.

A change of a crystal form of a pharmaceutical compound usually results in that the compound has different melting point, solubility, hygroscopicity, stability, bioactivity and the like, all of which would affect many important factors, such as ease of preparation, storage stability, ease of formulation, bioavailability and the like. If a compound has polymorphism, because a particular polymorph has a special thermodynamic property and stability, it is important to know a crystal form of a compound used in each dosage form in the process of preparation, thereby ensuring that a drug having the same morphology is used in the manufacture process. Therefore, it is necessary to ensure that a compound is a single crystal form or a known mixture of some crystal forms.

When judging which polymorph(s) is(are) preferable, many properties thereof must be compared, and a preferable polymorph is selected based on many physical properties. It is completely possible that a polymorph is preferable under some critical conditions, such as ease of preparation, stability, purity, hygroscopicity and the like. In other cases, different polymorphs may be preferable due to higher solubility or better pharmacokinetics.

The discovery of a new polymorph of a pharmaceutical compound provides an opportunity to improve physical properties of a drug, i.e. extending all properties of this substance, and thereby can better guide a study of the compound and a formulation thereof. Therefore, the sorafenib hemi-p-tosylate monohydrate crystal provided by the present invention has some advantages in at least one aspect of bioavailability, hygroscopicity, stability, solubility, purity, ease of preparation and the like, thereby achieving its commercial value in the manufacture of a drug and other applications.

### SUMMARY OF INVENTION

One aspect of the present invention provides a sorafenib hemi-p-tosylate monohydrate crystal having a structure represented by formula (11), characterized in that, in a X-ray powder diffraction pattern using Cu Kα irradiation, diffraction peaks occur at 2θ angle of about 5.62, 6.67, 8.05, 9.06, 9.63, 9.91, 10.95, 11.25, 13.48, 14.00, 14.60, 15.08, 15.75, 16.20, 16.62, 16.80, 17.23, 18.40, 18.97, 19.32, 19.82, 20.49, 20.74, 21.51, 22.56, 22.86, 23.37, 23.71, 24.20, 24.71, 24.97, 25.54, 25.80, 26.18, 27.14, 27.48 and 28.29 degree, preferably at 2θ angle of about 5.62, 6.67, 8.05, 9.06, 9.63, 9.91, 10.95, 11.25, 12.79, 13.48, 14.00, 14.60, 15.08, 15.75, 16.20, 16.62, 16.80, 17.23, 18.40, 18.97, 19.32, 19.82, 20.49, 20.74, 21.51, 22.06, 22.56, 22.86, 23.37, 23.71, 24.20, 24.71, 24.97, 25.54, 25.80, 26.18, 26.41, 27.14, 27.48, 28.29, 28.58, 29.15 and 29.88 degree, and more preferably at 2θ angle of about 5.62, 6.67, 8.05, 9.06, 9.63, 9.91, 10.95, 11.25, 12.79, 13.48, 14.00, 14.60, 15.08, 15.75, 16.20, 16.62, 16.80, 17.23, 18.40, 18.97, 19.32, 19.82, 20.49, 20.74, 21.51, 22.06, 22.56, 22.86, 23.37, 23.71, 24.20, 24.71, 24.97, 25.54, 25.80, 26.18, 26.41, 27.14, 27.48, 28.29, 28.58, 29.15, 29.88, 30.44, 31.20, 32.04, 32.67, 33.56, 34.07, 34.84, 36.32, 36.73, 37.31, 38.20, 38.87, 39.56, 40.44, 41.69, 43.47 and 44.28 degree.

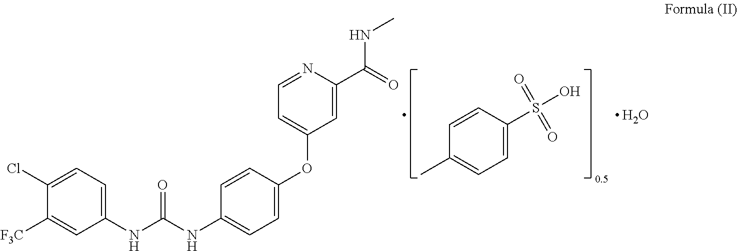

Further, in the X-ray powder diffraction pattern using Cu Kα irradiation of the sorafenib hemi-p-tosylate monohydrate crystal of the present invention, characteristic peaks have the following peak positions and intensities as shown in Table 1:

TABLE 1

| | | Relative |
|---|---|---|
| No. | 2θ (°) | Intensity (I/I₀) |
| 1 | 5.62 | 4.37 |
| 2 | 6.67 | 18.06 |
| 3 | 8.05 | 6.38 |
| 4 | 9.06 | 42.54 |
| 5 | 9.63 | 31.65 |
| 6 | 9.91 | 11.29 |
| 7 | 10.95 | 20.68 |
| 8 | 11.25 | 9.89 |
| 9 | 12.79 | 2.54 |
| 10 | 13.48 | 97.49 |
| 11 | 14.00 | 71.33 |
| 12 | 14.60 | 10.25 |
| 13 | 15.08 | 8.35 |
| 14 | 15.75 | 16.49 |
| 15 | 16.20 | 9.78 |
| 16 | 16.62 | 35.38 |
| 17 | 16.80 | 37.60 |
| 18 | 17.23 | 88.75 |
| 19 | 18.40 | 33.69 |
| 20 | 18.97 | 15.99 |
| 21 | 19.32 | 26.99 |
| 22 | 19.82 | 37.20 |
| 23 | 20.49 | 100.00 |
| 24 | 20.74 | 53.98 |
| 25 | 11.51 | 48.39 |
| 26 | 22.06 | 2.76 |
| 27 | 22.56 | 16.77 |
| 28 | 22.86 | 31.61 |
| 29 | 23.37 | 20.47 |
| 30 | 23.71 | 56.34 |
| 31 | 24.20 | 36.02 |
| 32 | 24.71 | 28.24 |
| 33 | 24.97 | 53.51 |
| 34 | 25.54 | 17.92 |
| 35 | 25.80 | 25.16 |
| 36 | 26.18 | 9.82 |
| 37 | 26.41 | 3.94 |
| 38 | 27.14 | 74.09 |
| 39 | 27.48 | 37.03 |
| 40 | 28.29 | 24.12 |

Further, in the X-ray powder diffraction pattern using Cu Kα irradiation of the sorafenib hemi-p-tosylate monohydrate crystal of the present invention, characteristic peaks have the following peak positions and intensities as shown in Table 2:

TABLE 2

| | | Relative |
|---|---|---|
| No. | 2θ (°) | Intensity (I/I₀) |
| 1 | 5.62 | 4.37 |
| 2 | 6.67 | 18.06 |
| 3 | 8.05 | 6.38 |
| 4 | 9.06 | 42.54 |
| 5 | 9.63 | 31.65 |
| 6 | 9.91 | 11.29 |
| 7 | 10.95 | 20.68 |
| 8 | 11.25 | 9.89 |
| 9 | 12.79 | 2.54 |
| 10 | 13.48 | 97.49 |
| 11 | 14.00 | 71.33 |
| 12 | 14.60 | 10.75 |
| 13 | 15.08 | 8.35 |
| 14 | 15.75 | 16.49 |
| 15 | 16.20 | 9.78 |
| 16 | 16.62 | 35.38 |
| 17 | 16.80 | 37.60 |
| 18 | 17.23 | 88.75 |
| 19 | 18.40 | 33.69 |
| 20 | 18.97 | 15.99 |
| 21 | 19.32 | 26.99 |
| 22 | 19.82 | 37.20 |
| 23 | 20.49 | 100.00 |
| 24 | 20.74 | 53.98 |
| 25 | 21.51 | 48.39 |
| 26 | 22.06 | 2.76 |
| 27 | 72.56 | 16.77 |
| 28 | 22.86 | 31.61 |
| 29 | 23.37 | 20.47 |
| 30 | 23.71 | 56.34 |
| 31 | 24.20 | 36.02 |
| 32 | 24.71 | 28.24 |
| 33 | 24.97 | 53.51 |
| 34 | 25.54 | 17.92 |
| 35 | 25.80 | 25.16 |
| 36 | 26.18 | 9.82 |
| 37 | 26.41 | 3.94 |
| 38 | 27.14 | 74.09 |
| 39 | 27.48 | 37.03 |
| 40 | 28.79 | 24.12 |
| 41 | 28.58 | 7.28 |
| 42 | 29.15 | 11.72 |
| 43 | 29.88 | 11.08 |
| 44 | 30.44 | 8.35 |
| 45 | 31.19 | 8.21 |
| 46 | 32.04 | 8.21 |
| 47 | 32.67 | 12.65 |
| 48 | 33.56 | 8.85 |
| 49 | 34.07 | 15.99 |
| 50 | 34.84 | 12.08 |
| 51 | 36.37 | 8.75 |
| 52 | 36.73 | 9.61 |
| 53 | 37.31 | 6.45 |
| 54 | 38.20 | 3.87 |
| 55 | 38.87 | 5.95 |
| 56 | 39.56 | 4.91 |
| 57 | 40.44 | 4.27 |
| 58 | 41.69 | 4.09 |
| 59 | 43.47 | 4.84 |
| 60 | 44.28 | 3.69 |

In a specific embodiment, the sorafenib hemi-p-tosylate monohydrate crystal of the present invention is characterized by the X-ray powder diffraction (XRD) pattern as shown in FIG. 1.

In a specific embodiment, the differential scanning calorimetry (DSC) of the sorafenib hemi-p-tosylate monohydrate crystal of the present invention shows an absorption peak at about 144.61° C. In particular, the sorafenib hemi-p-tosylate monohydrate crystal of the present invention is characterized by the differential scanning calorimetry (DSC) pattern as shown in FIG. 2.

In a specific embodiment, the sorafenib hemi-p-tosylate monohydrate crystal of the present invention is characterized by the thermogravimetric analysis (TGA) pattern as shown in FIG. 3.

In another aspect, the present invention provides a process for preparing sorafenib hemi-p-tosylate monohydrate crystal, comprising:

(1) mixing sorafenib with a mixed solvent of ethanol and water;

(2) adding p-toluenesulfonic acid or a hydrate thereof thereto;

(3) crystallizing and separating to obtain crystals, optionally drying the separated crystals.

A mass ratio of ethanol to water is 10-5:1, and in some specific embodiments of the present invention, the mass ratio of ethanol to water is 7.19:1.

A molar ratio of sorafenib to p-toluenesulfonic acid may be 1:0.5-1, and in some specific embodiments of the present invention, the molar ratio of sorafenib to p-toluenesulfonic acid is 1:0.54.

A temperature at step (1) is not higher than 30° C., and is preferably 20-30° C.

A temperature at step (2) is not higher than 30° C., and is preferably 20-30° C.

A temperature at step (3) is not higher than 30° C., and is preferably 20-30° C.

The mixing at step (1) may be performed under shaking or stirring.

In some specific embodiments of the present invention, the drying at step (3) is performed at a temperature of 60±5° C. under a vacuum condition.

In still another aspect, the present invention provides a crystal composition comprising the sorafenib hemi-p-tosylate monohydrate crystal, wherein the sorafenib hemi-p-tosylate monohydrate crystal accounts for 50% or more, preferably 80% or more, more preferably 90% or more, and most preferably 95% or more, by weight of the crystal composition.

In another aspect, the present invention provides a pharmaceutical composition comprising the sorafenib hemi-p-tosylate monohydrate crystal or a crystal composition thereof. The pharmaceutical composition comprises a therapeutically effective amount of the sorafenib hemi-p-tosylate monohydrate crystal of the present invention, or a crystal composition thereof. In addition, the pharmaceutical composition of the present invention may or may not comprise a pharmaceutically acceptable excipient. Furthermore, the pharmaceutical composition of the present application may further comprise one or more other therapeutic agents.

The “pharmaceutically acceptable excipient” refers to an inert substance which is administered together with an active ingredient, and facilitates the administration of the active ingredient, which includes, but is not limited to, any glidants, sweetening agents, diluents, preservatives, dyes/colorants, flavoring enhancers, surfactants, wetting agents, dispersing agents, disintegrating agents, suspending agents, stabilizing agents, isosmotic agents, solvents, or emulsifiers, which has been approved by the China Food and Drug Administration as being acceptable for use in humans or animals. The non-limiting example of the excipient includes calcium carbonate, calcium phosphate, various sugars and various starches, cellulose derivatives, gelatin, vegetable oils and polyethylene glycol.

The pharmaceutical composition of the present application can be formulated into a solid, hemisolid, liquid or gaseous formulation, such as tablets, pills, capsules, powders, granules, ointments, emulsions, suspensions, solutions, suppositories, injections, inhalants, gels, microspheres, aerosols and the like.

The typical administration route of the pharmaceutical composition of the present application includes, but is not limited to, oral, rectal, transmucosal, enteral administration, or topical, transdermal, inhalation, parenteral, sublingual, intravaginal, intranasal, intraocular, intraperitoneal, intramuscular, subcutaneous, intravenous administration. The preferable administration route is oral administration.

In another aspect, the present invention provides a use of the sorafenib hemi-p-tosylate monohydrate crystal, or a crystal composition thereof, or a pharmaceutical composition thereof, or a pharmaceutical composition comprising a crystal composition thereof in the preparation of a medicament for treating and/or preventing a cancer. Preferably, the cancer is renal cell carcinoma or hepatocellular carcinoma.

In yet another aspect, the present invention provides a method for treating and/or preventing a disease of a mammal (such as a human), comprising administering to the mammal (such as a human) a therapeutically effective amount of the sorafenib hemi-p-tosylate monohydrate crystal, or a crystal composition thereof, or a pharmaceutical composition thereof, or a pharmaceutical composition comprising a crystal composition thereof, wherein the disease is a cancer, preferably renal cell carcinoma or hepatocellular carcinoma.

In the present invention, X-ray powder diffraction spectrometric measurement is performed with the instrument model of Bruker D8ADVANCE ray diffractometer under the following conditions: Cu-Kα (voltage of 40 kV, and current of 40 mA), scanning range: 3-45°, scanning rate: 8°/min, step-size: 0.02°.

In the present invention, DSC spectrometric measurement is performed with the instrument model of METTLER TOLEDO DSC1 under the following conditions: a temperature rises at a rate of 10° C./min within a range of 50-300° C. to scan the DSC pattern.

In the present invention, TGA spectrometric measurement is performed with the instrument model of Netzsch TG 209 F1 Model thermogravimetric analyzer under the following conditions: temperature rises at a rate of 10° C./min within a range of 30-350° C. to scan the TGA pattern.

In the present invention, elemental analysis for C, H, and N elements is performed with the instrument of Carlo Erba Strumen-tasione Elemental Analyzer (MOD-1106), and a measurement for S, Cl, and F elements is performed with an oxygen flask combustion method.

As for any given crystal form, it is well-known in the field of crystallography that a relative intensity of a diffraction peak may change due to a preferred orientation caused by some factors, such as a crystal morphology and so on. Peak intensity will change at a position where a preferred orientation occurs, but the position of a characteristic peak for a crystal form will not change. Therefore, the relative intensity of the diffraction peak is not characteristic for the corresponding crystal form. When judging whether the given crystal form is identical to a known crystal form, the relative positions of the diffraction peaks should be noted, rather than relative intensities thereof. In addition, as for any given crystal form, it is also well-known in the field of crystallography that the position of a peak may have a slight error. For example, due to a change of a temperature, a movement of a sample, a calibration of an instrument and the like upon analyzing a sample, the position of a peak may shift, and accordingly a measurement error of 2θ value is ±0.2θ. Therefore, where determining each crystal structure, such error should be considered. In XRD pattern, the position of a peak is typically represented by 2θ angle or interplanar spacing d. The 2θangle and the interplanar spacing d have a simple conversion relation: d=λ/2 sin θ, wherein d represents interplanar spacing, λ represents wavelength of incident X ray, and θ represents diffraction angle.

The sorafenib hemi-p-tosylate monohydrate crystal of the present invention has a high stability, a low hygroscopicity, a high purity, and a high degree of crystallinity. In the meantime, the process for preparing the sorafenib hemi-p-tosylate monohydrate crystal of the present invention has some advantages, such as a simple and easy operation, a cheap and available solvent, a mild crystallization condition, and thereby is particularly suitable for industrial production.

### DESCRIPTION OF DRAWINGS

FIG. 1 shows an X-ray powder diffraction pattern of the sorafenib hemi-p-tosylate monohydrate crystal prepared in Example 1.

FIG. 2 shows a DSC pattern of the sorafenib hemi-p-tosylate monohydrate crystal prepared in Example 1.

FIG. 3 shows a TGA pattern of the sorafenib hemi-p-tosylate monohydrate crystal prepared in Example 1.

### SPECIFIC EMBODIMENTS

The following specific examples are only used to make a person skilled in the art more clearly understand and practice the present invention. They should not be considered to limit the scope of the present invention, and are only illustrations and representative examples for the present invention.

### Example 1: Preparation of Sorafenib Hemi-p-Tosylate Monohydrate

At a room temperature, to a reaction tank were added anhydrous ethanol (85.6 kg) and purified water (11.9 kg), and then sorafenib (10.84 kg, 23.32 mol) were added under stirring. After stirring for 10 min, p-toluenesulfonic acid monohydrate (2.38 kg, 12.51 mol) was added at one time. The resulting mixture was crystallized for 8 hours under stirring, and then filtrated under centrifugation. The resulting solid was dried for 10 hours at a temperature of 6035° C. under vacuum to obtain sorafenib hemi-p-tosylate monohydrate (11.48 kg, yield: 89.5% and purity: 99.96%).

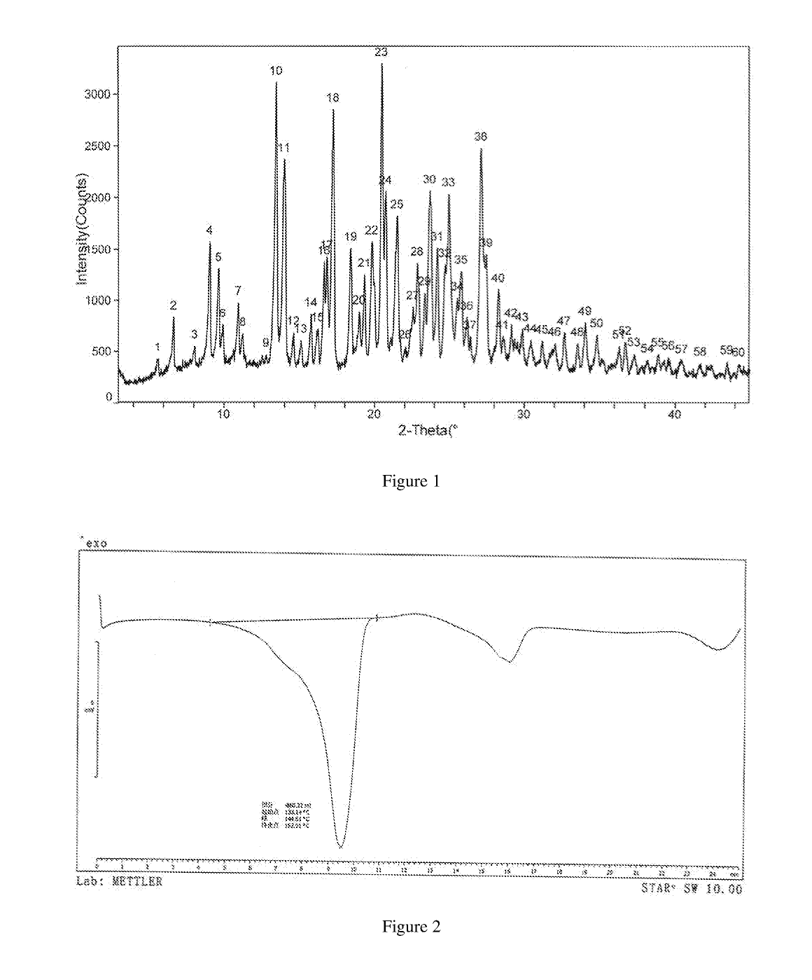

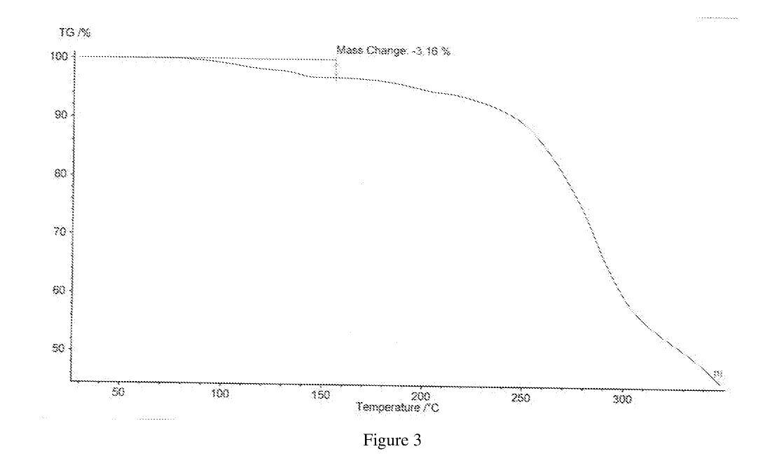

Its X-ray powder diffraction pattern using Cu Kα irradiation was shown in FIG. 1, differential scanning calorimetry (DSC) pattern was shown in FIG. 2, and thermogravimetric analysis (TGA) pattern was shown in FIG. 3.

Element analysis: C: 51.64% (theoretical value: 51.72%), H: 3.98% (theoretical value: 3.90%), N: 9.83% (theoretical value: 9.85%), S: 2.85% (theoretical value: 2.82%), Cl: 6.29% (theoretical value: 6.23%), F: 10.08% (theoretical value: 10.02%).

### Example 2: Liquid Phase Condition for HPLC Analysis of Sorafenib Hemi-p-Tosylate Monohydrate

Chromatographic column: Agilent peptide map chromatographic column (3.0×150 mm, 2.7 μm)

Mobile phase A: phosphate buffer solution (monopotassium phosphate (0.79 g) was weighted and dissolved in water, then diluted to 1000 ml, and further adjusted to pH=2.4 with phosphoric acid)

Mobile phase B: ethanol-acetonitrile (40:60)

Detection wavelength: 235 nm for detection

Flow rate: 0.6 ml/min

Column temperature: 55° C.

Injection volume: 10 μl

Solvent: mobile phase A-mobile phase B (1:3)

Preparation of a solution of a test sample: an appropriate amount of the test sample was weighted and dissolved in the solvent [mobile phase A-mobile phase B (1:3)], then diluted to a solution which comprises about 0.16 mg of sorafenib per 1 ml as the solution of the test sample.

A linear gradient elution was performed according to the program as shown in Table 3:

TABLE 3
Test condition for HPLC

| Time (min) | Mobile phase A (%) | Mobile phase B (%) |
|---|---|---|
| 0 | 80 | 10 |
| 12 | 56.5 | 43.5 |
| 40 | 10 | 90 |
| 45 | 80 | 10 |
| 55 | 80 | 20 |

### Example 3: Stability Test

The stability test for the sorafenib hemi-p-tosylate monohydrate crystal of the present invention was conducted according to the Chinese Pharmacopoeia (2010), Part 11, Appendix XIX C: Guideline for Stability Test of Bulk Drug and Pharmaceutical Preparation. The results were shown in Table 4.

TABLE 4
Results of stability test

| | | | | | Exposure test | | |
|---|---|---|---|---|---|---|---|
| | | | | | to light | Shading test | Standing for 6 |
| | | Standing | Standing | Standing for 10 | (luminance: | (luminance: | months at 25° C. ± |
| | | for 10 | for 10 | days at 25° C. | 6000 Lux), | 6000 Lux), | 2° C. and |
| | | days at | days at | and humidity of | standing for | standing for | humidity of |
| | Day 0 | 40° C. | 60° C. | 92.5% ± 5% | 10 days | 10 days | 60% ± 5% |
| Purity | 99.96% | 99.96% | 99.95% | 99.96% | 99.97% | 99.96% | 99.96% |

The results in the above table showed that the sorafenib hemi-p-tosylate monohydrate of the present invention is highly stable, and therefore particularly suitable for a pharmaceutical preparation.

### Example 4: Hygroscopicity Test

The hygroscopicity test for the sorafenib hemi-p-tosylate monohydrate crystal of the present invention was conducted according to the Chinese Pharmacopoeia (2010), Part II, Appendix XIX J: Guideline for Hygroscopicity Test of Drug. The hygroscopic weight grain of the sample was calculated, and the results were shown in Table 5.

TABLE 5
Results of hygroscopicity test

| Compound | Hygroscopic weight grain (%) |
|---|---|
| Sorafenib hemi-p-tosylate | 0.07 |
| monohydrate crystal | |

1. A sorafenib hemi-p-tosylate monohydrate crystal, characterized in that, in a X-ray powder diffraction pattern using Cu Kα irradiation, diffraction peaks occur at 2θ angle of 5.62, 6.67, 8.05, 9.06, 9.63, 9.91, 10.95, 11.25, 13.48, 14.00, 14.60, 15.08, 15.75, 16.20, 16.62, 16.80, 17.23, 18.40, 18.97, 19.32, 19.82, 20.49, 20.74, 21.51, 22.56, 22.86, 23.37, 23.71, 24.20, 24.71, 24.97, 25.54, 25.80, 26.18, 27.14, 27.48 and 28.29 degree.

2. The crystal of claim 1, characterized in that, in the X-ray powder diffraction pattern using Cu Kα irradiation, the diffraction peaks occur at 2θ angle of 5.62, 6.67, 8.05, 9.06, 9.63, 9.91, 10.95, 11.25, 12.79, 13.48, 14.00, 14.60, 15.08, 15.75, 16.20, 16.62, 16.80, 17.23, 18.40, 18.97, 19.32, 19.82, 20.49, 20.74, 21.51, 22.06, 22.56, 22.86, 23.37, 23.71, 24.20, 24.71, 24.97, 25.54, 25.80, 26.18, 26.41, 27.14, 27.48, 28.29, 28.58, 29.15 and 29.88 degree.

3. The crystal of claim 2, characterized in that, in the X-ray powder diffraction pattern using Cu Kα irradiation, the diffraction peaks occur at 2θ angle of 5.62, 6.67, 8.05, 9.06, 9.63, 9.91, 10.95, 11.25, 12.79, 13.48, 14.00, 14.60, 15.08, 15.75, 16.20, 16.62, 16.80, 17.23, 18.40, 18.97, 19.32, 19.82, 20.49, 20.74, 21.51, 22.06, 22.56, 22.86, 23.37, 23.71, 24.20, 24.71, 24.97, 25.54, 25.80, 26.18, 26.41, 27.14, 27.48, 28.29, 28.58, 29.15, 29.88, 30.44, 31.20, 32.04, 32.67, 33.56, 34.07, 34.84, 36.32, 36.73, 37.31, 38.20, 38.87, 39.56, 40.44, 41.69, 43.47 and 44.28 degree.

4. The crystal of claim 3, characterized in that, in the X-ray powder diffraction pattern using Cu Kα irradiation, characteristic peaks have positions and intensities as shown in the following table:

| | | Relative |
|---|---|---|
| No. | 2θ (°) | intensity (I/I₀) |
| 1 | 5.62 | 4.37 |
| 2 | 6.67 | 18.06 |
| 3 | 8.05 | 6.38 |
| 4 | 9.06 | 42.54 |
| 5 | 9.63 | 31.65 |
| 6 | 9.91 | 11.29 |
| 7 | 10.95 | 20.68 |
| 8 | 11.25 | 9.89 |
| 9 | 12.79 | 2.54 |
| 10 | 13.48 | 97.49 |
| 11 | 14.00 | 71.33 |
| 12 | 14.60 | 10.25 |
| 13 | 15.08 | 8.35 |
| 14 | 15.75 | 16.49 |
| 15 | 16.20 | 9.78 |
| 16 | 16.62 | 35.38 |
| 17 | 16.80 | 37.60 |
| 18 | 17.23 | 88.75 |
| 19 | 18.40 | 33.69 |
| 20 | 18.97 | 15.99 |
| 21 | 19.32 | 26.99 |
| 22 | 19.82 | 37.20 |
| 23 | 20.49 | 100.00 |
| 24 | 20.74 | 53.98 |
| 25 | 21.51 | 48.39 |
| 26 | 22.06 | 2.76 |
| 27 | 22.56 | 16.77 |
| 28 | 22.86 | 31.61 |
| 29 | 23.37 | 20.47 |
| 30 | 23.71 | 56.34 |
| 31 | 24.20 | 36.02 |
| 32 | 24.71 | 28.24 |
| 33 | 24.97 | 53.51 |
| 34 | 25.54 | 17.92 |
| 35 | 25.80 | 25.16 |
| 36 | 26.18 | 9.82 |
| 37 | 26.41 | 3.94 |
| 38 | 27.14 | 74.09 |
| 39 | 27.48 | 37.03 |
| 40 | 28.29 | 24.12. |

5. The crystal of claim 4, characterized in that, in the X-ray powder diffraction pattern using Cu Kα irradiation, the characteristic peaks have the positions and intensities as shown in the following table:

| | | Relative |
|---|---|---|
| No. | 2θ (°) | intensity (I/I₀) |
| 1 | 5.62 | 4.37 |
| 2 | 6.67 | 18.06 |
| 3 | 8.05 | 6.38 |
| 4 | 9.06 | 42.54 |
| 5 | 9.63 | 31.65 |
| 6 | 9.91 | 11.29 |
| 7 | 10.95 | 20.68 |
| 8 | 11.25 | 9.89 |
| 9 | 12.79 | 2.54 |
| 10 | 13.48 | 97.49 |
| 11 | 14.00 | 71.33 |
| 12 | 14.60 | 10.25 |
| 13 | 15.08 | 8.35 |
| 14 | 15.75 | 16.49 |
| 15 | 16.20 | 9.78 |
| 16 | 16.62 | 35.38 |
| 17 | 16.80 | 37.60 |
| 18 | 17.23 | 88.75 |
| 19 | 18.40 | 33.69 |
| 20 | 18.97 | 15.99 |
| 21 | 19.32 | 26.99 |
| 22 | 19.82 | 37.20 |
| 23 | 20.49 | 100.00 |
| 24 | 20.74 | 53.98 |
| 25 | 21.51 | 48.39 |
| 26 | 22.06 | 2.76 |
| 27 | 22.56 | 16.77 |
| 28 | 22.86 | 31.61 |
| 29 | 23.37 | 20.47 |
| 30 | 23.71 | 56.34 |
| 31 | 24.20 | 36.02 |
| 32 | 24.71 | 28.24 |
| 33 | 24.97 | 53.51 |
| 34 | 25.54 | 17.92 |
| 35 | 25.80 | 25.16 |
| 36 | 26.18 | 9.82 |
| 37 | 26.41 | 3.94 |
| 38 | 27.14 | 74.09 |
| 39 | 27.48 | 37.03 |
| 40 | 28.29 | 24.12 |
| 41 | 28.58 | 7.28 |
| 42 | 29.15 | 11.72 |
| 43 | 29.88 | 11.08 |
| 44 | 30.44 | 8.35 |
| 45 | 31.19 | 8.21 |
| 46 | 32.04 | 8.21 |
| 47 | 32.67 | 12.65 |
| 48 | 33.56 | 8.85 |
| 49 | 34.07 | 15.99 |
| 50 | 34.84 | 12.08 |
| 51 | 36.32 | 8.75 |
| 52 | 36.73 | 9.61 |
| 53 | 37.31 | 6.45 |
| 54 | 38.20 | 3.87 |
| 55 | 38.87 | 5.95 |
| 56 | 39.56 | 4.91 |
| 57 | 40.44 | 4.27 |
| 58 | 41.69 | 4.09 |
| 59 | 43.47 | 4.84 |
| 60 | 44.28 | 3.69. |

6. The crystal of claim 1, characterized substantially by the X-ray powder diffraction pattern as shown in FIG. 1.

7. The crystal of claim 1, having an absorption peak at 144.61° C. in a DSC pattern.

8. The crystal of claim 7, characterized by the differential scanning calorimetry pattern as shown in FIG. 2.

9. The crystal of claim 1, characterized by a thermogravimetric analysis pattern as shown in FIG. 3.

10. A process for preparing the crystal of claim 1, comprising:
(1) mixing sorafenib with a mixed solvent of ethanol and water,
(2) adding p-toluenesulfonic acid or a hydrate thereof thereto, and
(3) crystallizing and separating to obtain the crystal.

11. The process of claim 10, wherein a mass ratio of ethanol to water is 10-5:1.

12. The process of claim 11, wherein the mass ratio of ethanol to water is 7.19:1.

13. The process of claim 10, wherein a molar ratio of sorafenib to p-toluenesulfonic acid is 1:0.5-1.

14. The process of claim 13, wherein the molar ratio of sorafenib to p-toluenesulfonic acid is 1:0.54.

15. The process of claim 10, wherein a temperature at step (1), (2) or (3) is not higher than 30° C.

16. A crystal composition, comprising the crystal of claim 1, wherein the crystal accounts for 50% or more by weight of the crystal composition.

17. A pharmaceutical composition, comprising the crystal of claim 1.

18. A method for treating a cancer, comprising administering the crystal of claim 1 to a subject in need thereof.

19. A pharmaceutical composition, comprising the crystal composition of claim 16.

20. A method for treating a cancer, comprising administering the crystal composition of claim 16 to a subject in need thereof.
